(19) United States
(12) Patent Application Publication
(10) Pub. No.: US 2023/0277339 A1
(43) Pub. Date: Sep. 7, 2023

(54) RETROSPECTIVE TRAINING OF ADAPTIVE CONTROL SYSTEM FOR PROSTHETIC DEVICES

(71) Applicant: INFINITE BIOMEDICAL TECHNOLOGIES, LLC, Baltimore, MD (US)

(72) Inventors: Rahul Kaliki, Baltimore, MD (US); Ananth Natarajan, Incline Village, NV (US)

(21) Appl. No.: 18/116,136

(22) Filed: Mar. 1, 2023

Related U.S. Application Data
(60) Provisional application No. 63/315,948, filed on Mar. 2, 2022.

(51) Int. Cl. A61F 2/72

(52) U.S. Cl. CPC A61F 2/72; A61F 2002/6827

(57) ABSTRACT

Embodiments are directed to a prosthetic system comprising a myoelectric controlled prosthetic device and an electronic device for receiving user feedback on operation of the prosthetic device. The prosthetic device can include one or more sensors configured to detect myoelectric signals and a controller configured to provide myoelectric data as input to a classification model. The controller can receive one or more movement classes from the classification model and cause one or more actuators to perform one or more movements based on the movement classes. The electronic device can include an input/output component that can receive an indication from the user of whether the classification model correctly identified the user's intended movement. In response to a user indicating that an incorrect movement was performed, the system can retroactively update the classification model based on a correct movement that was identified by the user.

### CROSS-REFERENCE TO RELATED APPLICATION

This application is a nonprovisional patent application of and claims the benefit under 35 U.S.C. § 119(e) of U.S. Provisional Patent Application No. 63/315,948, filed Mar. 2, 2022, and titled “Retrospective Training of Adaptive Control System for Prosthetic Devices”, the contents of which are incorporated herein by reference in its entirety.

### FIELD

The described embodiments relate generally to control systems for patient-worn electromechanical prosthetic devices and in particular to adaptive control systems for prosthetic devices.

### BACKGROUND

Prosthetic devices can be used by amputee patients to restore partial or complete limb function. A myoelectric prosthetic device can leverage electromyography to receive and interpret electrical signals, detected from electrodes positioned over a patient's skeletal musculature, as positioning or pose instructions that, in turn, can be used to drive one or more electromechanical actuators of the prosthetic device.

However, conventional sensing and control systems of conventional myoelectric prosthetic devices typically leverage linear classification algorithms to interpret myoelectric signaling as specific pose or position intent(s). Such conventional techniques require inconvenient, time consuming, and regular retraining to maintain performance. For example, electrode positioning may shift over time and/or certain limb or body positions or states (e.g., respiration, perspiration, hydration, patient movement and so on) may adversely affect signal detection, which can lead to misclassification of a patient's intent which, in turn, causes the prosthetic to perform an unintended movement, or to transition to an unintended pose.

### BRIEF DESCRIPTION OF THE DRAWINGS

Reference will now be made in detail to representative embodiments illustrated in the accompanying drawings. It should be understood that the following descriptions are not intended to limit the embodiments to one preferred embodiment. To the contrary, it is intended to cover alternatives, modifications, and equivalents as can be included within the spirit and scope of the described embodiments as defined by the appended claims.

In particular, the disclosure will be readily understood by the following detailed description in conjunction with the accompanying drawings, wherein like reference numerals designate like structural elements, and in which:

FIG. 1 depicts an example myoelectric prosthetic device that includes an adaptive control system, as described herein;

FIG. 2 depicts an example system diagram of an electronic device that may perform operations as described herein;

FIG. 3 depicts an example system diagram of an adaptive control system of a myoelectric prosthetic device as described herein;

FIGS. 4A-4C depict example user interfaces for providing patient input to an adaptive control system of a myoelectric prosthetic as described herein;

FIG. 5 is a flowchart depicting example operations of a method for determining a control command for a prosthetic device from sensor input data;

FIG. 6 is a flowchart depicting example operations of a method for updating a control system based on a user indicating an incorrect prosthetic movement;

FIG. 7 shows an example process for an example process for determining a movement sequence from myoelectric signals; and

FIG. 8 shows an example process for a user confirmation process prior to performing an identified movement.

It should be understood that the proportions and dimensions (either relative or absolute) of the various features and elements (and collections and groupings thereof) and the boundaries, separations, and positional relationships presented therebetween, are provided in the accompanying figures merely to facilitate an understanding of the various embodiments described herein and, accordingly, may not necessarily be presented or illustrated to scale, and are not intended to indicate any preference or requirement for an illustrated embodiment to the exclusion of embodiments described with reference thereto.

### DETAILED DESCRIPTION

Embodiments described herein are directed to an adaptive prosthetic system that can perform retrospective training to dynamically update prosthetic control models.

The systems and methods described herein leverage machine learning models to identify an intended patient (also may be referred to as a “user” or “wearer” or “operator” of a prosthetic device) movement from detected myoelectric signals.

The machine learning model(s) can be updated and/or retrained, retrospectively by the patient while wearing the prosthetic. More specifically, if the prosthetic performs an unintended movement or transitions to an unintended pose, the patient can provide input to the prosthetic (and/or another electronic device, such as a cellular phone or smart watch) that the most-recently performed action incorrectly classified the patient's intent. The patient may also provide input indicating the intended action or pose not performed by the device. Once patient input is received, the prosthetic device can access a buffer, log, or other database to retrieve a representation of the myoelectric signals that were misinterpreted. These signals can be relabeled with the patient's intended action, and can be added to a training dataset or dictionary used to train the machine learning model(s). Thereafter, the machine learning models can be retrained against the entire training dataset (or, in some cases, a portion thereof) so that the unintended movement or pose experienced by the patient is less likely to occur again.

In some embodiments, prior training data determined (e.g., via k-means clustering in one example) to have influenced the incorrect intent classification may be removed from the training dataset prior to retraining of the machine learning models.

As a result of these and other described constructions, a myoelectric prosthetic can adjust, in the field while being worn by a patient, to changing conditions that otherwise, as noted above, may over time cause detected myoelectric signals to be incorrectly classified.

In this manner, more generally and broadly, the systems and methods described herein relate to adaptive and retrospective techniques that provide robust myoelectric signal classification and differentiation to determine an intended patient movement or pose for a prosthetic device in continually changing environmental conditions. These adaptive control techniques may improve the performance of a patient's control over a prosthetic device, such as an electromechanical prosthetic hand. In addition, the systems and methods described herein may result in reduced rejection rates and reduced instances of discontinued prosthetic use.

For simplicity of description, the embodiments described herein reference externally worn prosthetics (which may also be referred to as exoprostheses), but it may be appreciated that this is merely one example of a myoelectric prosthetic that can leverage the systems and methods described herein. For example, some embodiments described herein can apply equivalently to endoprostheses, implanted biometric devices, non-prosthetic human-machine interface devices, and so on. Further, for simplicity of description many embodiments that follow reference a prosthetic configured for use by a patient with an upper limb amputation, such as a transhumeral amputation or a transradial amputation. However, it may be appreciated that these are merely examples and other prosthetic devices can leverage the systems and methods described herein; these examples are not exhaustive.

Generally and broadly, as noted above, a patient may provide instructions to a myoelectric prosthetic device by activating skeletal muscles and/or nerves in a residual limb and/or surrounding areas or other locations (e.g., chest, back, and so on). The resulting electrical signals, generated by the patient's peripheral nervous system, can be detected by one or more electrodes (also referred to herein as “sensors” or “myoelectric sensors”) positioned on the patient's skin and/or implanted within the patient's musculature. A patient can generate different patterns of myoelectric signals that can be associated to specific movements or poses of the myoelectric prosthetic.

Aa calibration/training procedure can be performed to correlate different myoelectric patterns generated by a patient with specific movements or poses that the prosthetic device can perform or transition to. The calibration procedure can be leveraged to train a classification model, which in turn can be used to control operation of the prosthetic device.

For example, when a patient intends their prosthetic to perform a specific action, the patient can cause to be generated a unique electric pattern which can be sampled by a set or array of electrodes/sensors. Output from the sensors can be provided as input to the classification model. The classification model can provide, as output, a movement or pose associated with the input signal. The pose or movement can thereafter be used by an electromechanical control system of the prosthetic to change an angular and/or linear position of one or more electromechanical actuators of the prosthetic, thereby moving the prosthetic and/or changing a pose thereof.

Traditional myoelectric controlled prosthetics are calibrated by attaching myoelectric sensors to a patient, and requiring the patient to perform a series of tasks, which in turn are used to build a static classification model. The static classification model remains fixed until a new calibration procedure is performed, and thus cannot adapt over time to changing environmental or patient conditions. For example, the myoelectric signals detected by the sensors may shift due to movement of one or more of the sensors from a calibrated position.

Additionally or alternatively, changes in the patient's posture, fatigue, load on the limb, skin-electrode impedance, and so on may also cause a shift in the detected myoelectric signals. These shifts may cause the calibration of the conventional static classification model to become inaccurate over time, leading to performance degradation of the prosthetic device while in use. Phrased in another manner, the static classification model may increasingly mis-classify myoelectric signals, causing the prosthetic device to perform movements or transition to poses that are not intended by a patient.

One conventional solution to loss of calibration is to reperform a calibration procedure. However, as noted above, recalibration can be time consuming and may interrupt a patient's schedule. Notably, in highly dynamic conditions, a patient may be required to recalibrate a prosthetic device numerous times a day, significantly reducing the utility thereof.

By contrast, the systems and methods described herein include an adaptive model that can be updated in real-time using feedback from a patient. In particular, in some embodiments, after a myoelectric pattern has been classified, and the system has identified an intended gesture, movement, or pose to be performed, the system may output an indication of the identified gestured to the patient. The patient may confirm whether or not the correct gesture was identified. In response to the system identifying an a gesture that was different from what the patient intended, the system can leverage the previously-received myoelectric data associated with the misidentified gesture to updated the classification model.

In some cases, this operation can include receiving an indication from the patient of the correct intendent gesture, and retraining the classification model so that the myoelectric data that resulting in an incorrect gesture classification now results in a gesture classification intended by the patient.

In this manner, a system as described herein may adapt in real-time based on feedback from the patient while in use. Notably, these adaptations can be performed on a portion of the calibration model (e.g., only the model data associated with the misclassified gesture), which can allow retraining operations to be rapidly and seamlessly integrated into the control system without needing to reperform an entire calibration procedure.

Generally and broadly, a classification model as described herein can include (1) a machine learning component that is trained to classify detected myoelectric signals as an intended patient motion, pose, or gesture for a prosthetic device and (2) a training component that uses patient feedback to update the machine learning component.

In some cases, the machine learning component can include an extreme learning machine (ELM) that receives as input a vector corresponding to detected myoelectric signal data and provides as output one or more classifications of that input and a confidence (also referred to as an activation strength or simply an “activation”) associated with each classification. The one or more classifications can correspond to a movement/pose/gesture that the patient intends the prosthetic device to perform. Accordingly, in some cases, the control system can output control signals that cause a prosthetic device to perform a specific movement based on the classification determined by the ELM.

Additionally or alternatively, the machine learning component can include a sparce representation classifier (SRC). The SRC may be used to distinguish between two or more similarly activated movement classifications as determined by the ELM. For example, the ELM may output two (or more) movement classifications that have similar confidence metrics. In these cases, the SRC can be leveraged to analyze the myoelectric data to determine which of the two movements was more likely intended by the patient. In some cases, the SRC may operate a limited data set for the two (or more) movement classifications identified by the ELM, which may increase a computational speed of the SRC determination of the movement classification. In these embodiments, the SRC may also consume a smaller memory footprint, as gestures easily classifiable by the ELM need not be included in the SRC dictionary.

The training component of a system as described herein can allow a patient of a prosthetic device to indicate whether the machine learning component correctly classified the myoelectric data. In some cases, the control system can cause the prosthetic device to perform a movement that is associated with the movement identified by the machine learning component.

The system can further include an output device that allows a patient to indicate whether the performed movement was incorrect. Accordingly, if the prosthetic device the patient may immediately indicate that an unintended movement was performed. Additionally or alternatively, the patient may indicate their intended movement to the prosthetic via the output device.

In particular, as noted above, a patient feedback indicating that an incorrect action was performed by the prosthetic, and an indication of the intended (correct) movement can be used to update the machine learning component. In some cases, this can include updating the data libraries associated with the SRC.

For example, if the ELM identified two potential movements as the intended movement and the SRC incorrectly output one of those movements as the intended movement, then the SRC data libraries (also referred to as the SRC “dictionary”) associated with the two movements can be updated to achieve the correct movement output. The SRC data libraries can be updated so that the same myoelectric data that originally lead to the incorrect movement classification now lead to the correct movement classification.

In some cases, an update operation as described above can include removing a portion of data stored in the SRC libraries for the two movements and updating this portion based on the detected myoelectric signals and the correct movement as indicated by the patient. In other cases, the data in the SRC libraries can be replaced, for example, by using the detected myoelectric data to regenerate library data for the correct movement.

Additionally or alternatively, the ELM can be updated based on the detected myoelectric data and the patient feedback. For example, the myoelectric data can be used to retrain the ELM classification model so that the detected myoelectric data is associated with the correct prosthetic movement that was indicated by the patient.

Using an ELM can have the advantage of quick retraining times allowing the ELM classification model to updated in real-time (e.g., in response to the patient feedback), while minimizing any downtown for the prosthetic system. Additionally, the use of the ELM to reduce the data set to a limited number of movement classifications allows the SRC to operate more quickly, to support real-time movement classification.

As one non-limiting example, the systems and methods described herein may be applied to a myoelectric upper limb prosthetic that includes an electromechanical hand. The system can include an array of sensors that are each positioned at a different location on a patient's residual limb or supporting musculature (e.g., chest, back, neck, and so on). The array of sensors can detect myoelectric signals that are generated by a patient when the patient wants the lower limb prosthetic to perform a particular movement or action by the prosthetic. The system can generate a dataset from the myoelectric signals and provide the dataset as input to the classification model. The output of the classification can be used to identify an intended patient movement and the control system can send control signals to the electromechanical hand to perform the movement identified by the classification model.

In additional to sending the control signals, the system can also output an indication of the identified movement to the patient. For example, the system can include a smartwatch with displays and indication of the movement performed by the electrotechnical hand and an option for the patient to correct the movement. Using an interface on the smartwatch the patient may indicate that the movement was not correct. Additionally or alternatively, the patient may select on the smart watch, a movement that she or he intended to perform.

The patient feedback, including an indication that the wrong movement was performed and an indication of the correct movement, can be sent to the control system to update the machine learning component as described herein. Accordingly, the next time the patient generates the same and/or similar myoelectric signals, the control system will classify these signals as corresponding to the movement designated by the patient.

Notably, the above-described operations can be performed in real time, which may eliminate a need for a patient to perform time consuming recalibration operations in response to an incorrect intent classification. The retrospective calibration operations described herein may further allow a patient to continually train and update their prosthetic system account for environmental changes such as movement of the sensors, different loading patterns on the arm, changes in sensor impedance, fatigue and/or other conditions that lead to misclassification of myoelectric signals.

In some cases, a control system as described herein can be configured to take contextual factors into account when determining an intended user movement. The contextual factors can include parameters such as a current limb position, a previous movement or sequence of movements, time of day, type of activity a user is engaged in, and so on. In some cases, contextual factors may be used to select a movement classification from two (or more) movement classifications identified by the classification model.

For example, a patient intending to grasp an object may have extend their arm and opened the prosthetic hand, in preparation for grabbing the object. The classification model (by operation of an ELM and/or SRC system, as described above) may then determine that the next instructed movement is either an extend index finger pose or a hand close operation.

In this example, the system may track and analyze use patterns over time and collect data indicating that that arm extension and palm open movements are highly likely to be followed by a hand close operation, and have lower correlation to movements that include extending an index finger. Accordingly, the system may preferentially select the close hand movement based on the contextual analysis indicating that the close hand operation is more likely than the index finger extension pose.

In other cases, the system may use contextual information as a factor, for example, by leveraging contextual information to bias an activation associated with a particular movement that has been identified by a classification model.

In other cases, the control system can be configured to analyze myoelectric signals to identify a sequence of movements that a user in intending to perform. In some cases, a user may perform a complex sequence of movements with their arm and/or hand in which different portions (joints) are simultaneously performing different movements. For example, a person who is going to shake someone's had may simultaneously (or near simultaneals) move their arm upward from their side while opening their fingers and rotating their wrist to position their hand in the correct orientation. For a limb amputee these signals may show up as a distinct pattern in their residual limb. Further, it may be had to pull one motion out of the myoelectric signals because the user is intending to simultaneously perform multiple movements.

Accordingly, the control system may be configured to recognize sequences of movements and generate control commands based on an intended sequence of movements. In some cases, the sampling period of the myoelectric sensor may be adjusted to facilitate capturing myoelectric signals corresponding to a movement sequence. For example, the sampling period can be implemented as a sliding window or other dynamic period that monitors the myoelectric activity and adjusts a length of the data collection to capture a complete signal sequence. The system can analyze and preprocess the measured myoelectric signals to generate an input data set.

In addition to or as an alternative to the discrete movement training data, the classification model can be trained to recognize movement sequences. In some cases, the trained movement sequences may include common movement combinations, such as shaking a hand, reaching out to grasp an object, and so on. Although these can be specific to each individual user. Accordingly, the myoelectric data may be input into the classification model, and the classification model may identify a movement sequence as having the highest probability of being in the intend movement. In these cases, the control system may generate control commands for the movement sequence. Additionally or alternatively, timing/steps of a movement sequence may be controlled using one or more sensors. For example, when performing a grabbing motion the user may extend their arm and the hand prothesis may move to an open position. However, the system may wait for feedback from a sensors, such as a pressure sensor on the hand, before closing the fingers around an object. Accordingly, the techniques and methods described herein may be used to identify combinations of movements that are indicated by a user through a unique combination of myoelectric signals.

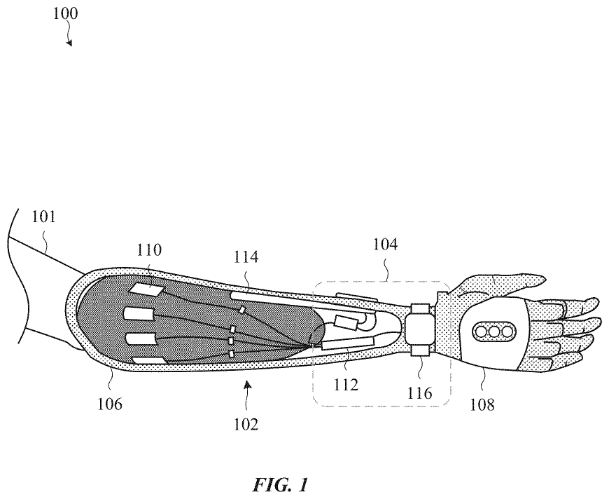

These and other embodiments are discussed below with reference to FIGS. 1-8. However, those skilled in the art will readily appreciate that the detailed description given herein with respect to these Figures is for explanatory purposes only and should not be construed as limiting.

FIG. 1 shows an example of a prosthetic system 100 including a prosthetic device 102 and an adaptive feedback system 104. The prosthetic device 102 can include a frame 106 that fits over a residual limb 101 of a patient. The frame 106 can couple to an electromechanical device 108, such as a prosthetic hand. In some cases, the electromechanical device 108 can include components that control movement and/or other functions of the device. For example, the electromechanical device 108 can include motors, sensors, a control system that controls movement of device and so on. In some cases, the electromechanical device 108 can include an independent power source. In other cases, the electromechanical device 108 can use a power source (e.g., power source 114) positioned on the frame 106.

The prosthetic device 102 can include one or more sensors 110 (one of which is labeled for clarity), a control system 112 and a power source 114. The control system 112 can exchange control commands and/or other data with the electromechanical device 108. For example, in cases where the electromechanical device 108 is a prosthetic hand, the control system 112 may send commends to the prosthetic hand that instruct the prosthetic hands to perform a specific movement such as opening the fingers of the hand. The prosthetic hand may receive the command and one or more onboard processors system can cause the hand to perform the requested function(s).

The sensors 110 can be configured to detect myoelectric signals at the patient's residual limb 101. The sensors 110 can include multiple electrodes that coupled to the frame 106 and contact the patient's residual limb 101 when the prosthetic device 102 is worn by the patient. The sensors 110 can be communicably coupled to the control system 112.

The power source 114 can be coupled to the frame 106, and in some cases, may be positioned on an inside portion of the frame. The power source 114 may be a flexible power source that can conform to an interior surface of the frame 106 and/or a portion of the patient's limb 101. The power source 114 can include a battery or other suitable power source as described herein and may be removed and/or rechanged.

In some cases, the frame 106 can include a coupling mechanism that allows different the electromechanical devices 108 to be removably coupled to the frame 106. Additionally, the coupling mechanism can allow other types of electromechanically devices to be coupled to the frame and/or the frame 106 can include different types of coupling mechanism that couple with other electromechanical device. Accordingly, the frame 106, the sensors 110, the control system 112 and the power source 114 may form a sub-prosthetic that can interface with different types of actuating prothesis. This sub-prothesis may primarily function to detect a patient's myoelectric signals, processes those signals to determine an intended movement, and send control commands to an electrotechnical device 108 for performing the intended movement.

The adaptive feedback system 104 can include the control system 112 and a patient device 116. The patient device 116 can be a wearable electronic device such as a smartwatch that is communicably coupled to the control system 112 via any suitable wireless transmission protocol. In some cases, the patient device 116 can be a smartphone, tablet, or other suitable portable electronic device electronic device. In yet other cases, the electronic device 116 can be integrated with the frame 106.

The patient device 116 can be configured to output information to the patient and receive feedback from the patient. In some cases, the patient device 116 can include a touch-sensitive display, audio, haptic and/or other output mechanism. The patient device 116 may also receive a variety of input types from a patient such as touch inputs (e.g., to the touch-sensitive display), speech inputs such as voice commands, and/or other suitable input types. The patient device 116 can transmit indications of received patient inputs to the control system 112. Accordingly, a patient may use the patient device 116 to provide feedback to the prosthetic device 102.

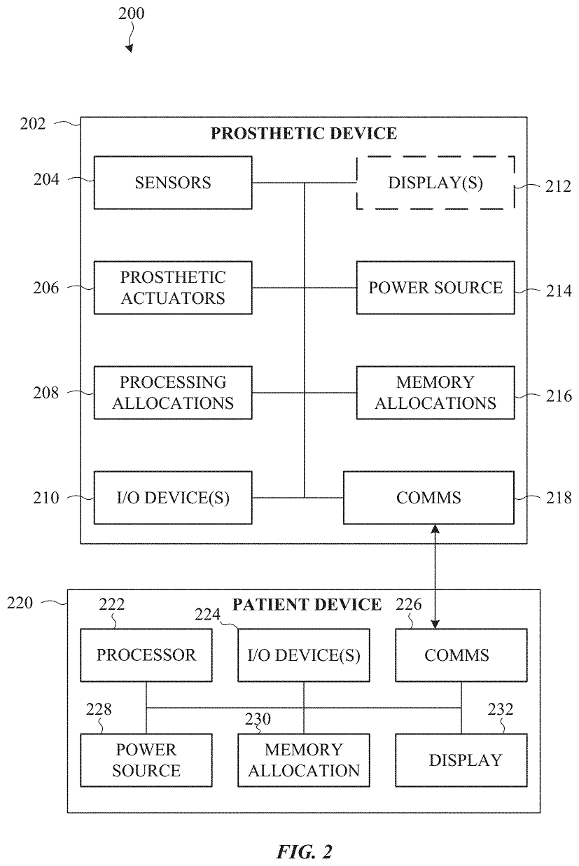

FIG. 2 shows an example electrical block diagram of a prosthetic system 200 that may perform the operations described herein. The prosthetic system 200 can have a prosthetic device 202, which can be an example of the prosthetic devices described herein. The prosthetic device 202 can include one or more sensors 204, one or more prosthetic actuators 206, processing allocations 208, input/output (I/O) devices 210, a display 212 (which may, in some examples, be optional like other components shown in FIG. 2), a power source 214, memory allocation 216, and communication devices (COMMS) 218. The prosthetic system 200 can also include a patient device 220 that is an example of the patient devices described herein. The patient device 220 can include a micro-processor 222, memory 224, communication devices (COMMS) 226, I/O devices 228 and a display 228.

The prosthetic system may also include one or more sensors 204 positioned at different locations on the prosthetic system 200. The sensor(s) 204 can be myoelectric sensors that are configured to sense myoelectric signals in a residual limb of a patient as described herein. Additionally, the system can include other sensors 204 configured to sense one or more types of parameters, such as but not limited to, pressure, light, touch, heat, movement, relative motion, biometric data (e.g., biological parameters), and so on.

For example, these additional sensor(s) 204 may include a thermal sensor, a position sensor, a light or optical sensor, an accelerometer, a pressure transducer, a gyroscope, a magnetometer, a health monitoring sensor, and so on. Additionally, the one or more sensors 204 can utilize any suitable sensing technology, including, but not limited to, capacitive, ultrasonic, resistive, optical, ultrasound, piezoelectric, and thermal sensing or imaging technology.

The prosthetic actuators 206 can include motors, hydraulic actuators, mechanical linkages, gear driven system and/or other electromechanical systems that create movement in one or more portions of the prosthetic device such as a prosthetic hand. The prosthetic actuators can include motors and assemblies that drive movement of one or more prosthetic fingers, wrist movement, other hand movement, and/or movements of other joints such as an elbow in the case of an upper arm amputation.

The processor allocations 208 can be implemented as any electronic device capable of processing, receiving, or transmitting data or instructions. For example, the processor allocations 208 can be a microprocessor, a central processing unit (CPU), an application-specific integrated circuit (ASIC), a digital signal processor (DSP), or combinations of such devices. As described herein, the term “processor” is meant to encompass a single processor or processing unit, multiple processors, multiple processing units, or other suitable computing element or elements. The processing unit can be programmed to perform the various aspects of the systems described herein.

It should be noted that the components of the prosthetic system 200 can be controlled by multiple processors. For example, select components of the prosthetic system 200 (e.g., a sensor 204) may be controlled by a first processor and other components of the prosthetic system 200 (e.g., the prosthetic actuators 206) may be controlled by a second processor, where the first and second processors may or may not be in communication with each other.

The I/O devices 210 can be any suitable mechanisms that allows a patient to provide input to prosthetic device 202 and/or the patient device 220 and receive feedback from these devices. In some cases, the I/O devices 210 can be touch- and/or force-sensitive and include or be associated with touch sensors and/or force sensors that extend along the output region of the display and which may use any suitable sensing elements and/or sensing techniques.

Using touch sensors, the prosthetic device 202 and/or patient device 220 may detect touch inputs applied to a display region including detecting locations of touch inputs, motions of touch inputs (e.g., the speed, direction, or other parameters of a gesture applied to the cover 154), or the like. Using force sensors, the prosthetic device 202 and/or patient device 220 may detect amounts or magnitudes of force associated with touch events applied to the cover 154. The touch and/or force sensors may detect various types of patient inputs to control or modify the operation of the device, including taps, swipes, multiple finger inputs, single- or multiple-finger touch gestures, presses, and the like.

Additionally or alternatively, the I/O devices 210 can include buttons, or other suitable input devices. In some cases, the I/O devices 210 can include a microphone and/or speaker and be configured to output sounds and receive voice-feedback. For example, a patient may be able to provide voice feedback via the patient device 220 on movement performed by their prosthetic device.

As noted above, the prosthetic system 200 may optionally include the display 212 such as a liquid-crystal display (LCD), an organic light emitting diode (OLED) display, a light emitting diode (LED) display, or the like. If the display 212 is an LCD, the display 212 may also include a backlight component that can be controlled to provide variable levels of display brightness. If the display 212 is an OLED or LED type display, the brightness of the display 212 may be controlled by modifying the electrical signals that are provided to display elements. The display 212 may correspond to any of the displays shown or described herein.

The power source 214 can be implemented with any device capable of providing energy to the prosthetic device 202. For example, the power source 214 may be one or more batteries or rechargeable batteries. Additionally or alternatively, the power source 214 can be a power connector or power cord that connects the prosthetic device 202 to another power source, such as a wall outlet.

The memory allocations 216 can store electronic data that can be used by the prosthetic device 202. For example, the memory 216 can store electrical data or content such as, for example, audio and video files, documents and applications, device settings and patient preferences, timing signals, control signals, and data structures or databases. The memory 216 can be configured as any type of memory. By way of example only, the memory 216 can be implemented as random access memory, read-only memory, Flash memory, removable memory, other types of storage elements, or combinations of such devices.

The communication device 218 can transmit and/or receive data from a patient or another electronic device. A communication device can transmit electronic signals via a communications network, such as a wireless and/or wired network connection. Examples of wireless and wired network connections include, but are not limited to, cellular, Wi-Fi, Bluetooth, IR, and Ethernet connections. In some cases, the communication device 218 can communicate with an external electronic device, such as a smartphone, electronic device, or other portable electronic device, as described here.

The patient device 220 can include a processor 222, I/0 devices 224, communication devices 226, a power source 228, memory allocations 230 and a display 232, which can be housed in an independent structure that functions independently of the prosthetic device 202.

In other cases, the patient device 220 can be configured to conductively couple to the prosthetic device 202, for example by a data and/or power cable.

The patient device 220 may be any suitable electronic device including, but not limited to a cellular phone, smart watch, tablet device, laptop computer, wearable device, and so on. These examples are not limiting; the patient device 220 can be any suitable electronic device.

The processor 222 of the patient device 220 can be configured to cooperate with the memory allocations 230 to instantiate one or more instances of software configured to perform, coordinate, or supervise one or more operations described herein. For example, in some embodiments, the patient device 220 can be configured to instantiate controller software configured to execute one or more classification or retraining operations, such as described above.

In other cases, the patient device 220 can be configured to instantiate an instance of software that records a timeline of events/actions performed by the prosthetic device 202. In these examples, the patient can operate the patient device 220 to select an action performed by the prosthetic at a known time, but not immediately in the past. For example, in some circumstances, the patient may not be immediately able to signal to the prosthetic device that a particular action or pose was incorrect. In these examples, the patient may leverage the patient device 220 to review actions (which may be tagged with time and/or geolocation) taken by the prosthetic, so that the patient can provide input indicating that one or more actions were not correct. In a more simple phrasing, in some embodiments, the patient device 220 can be used to correct any historical action taken by the prosthetic device, not just actions taken immediately preceding or near-in-time with the patient's input.

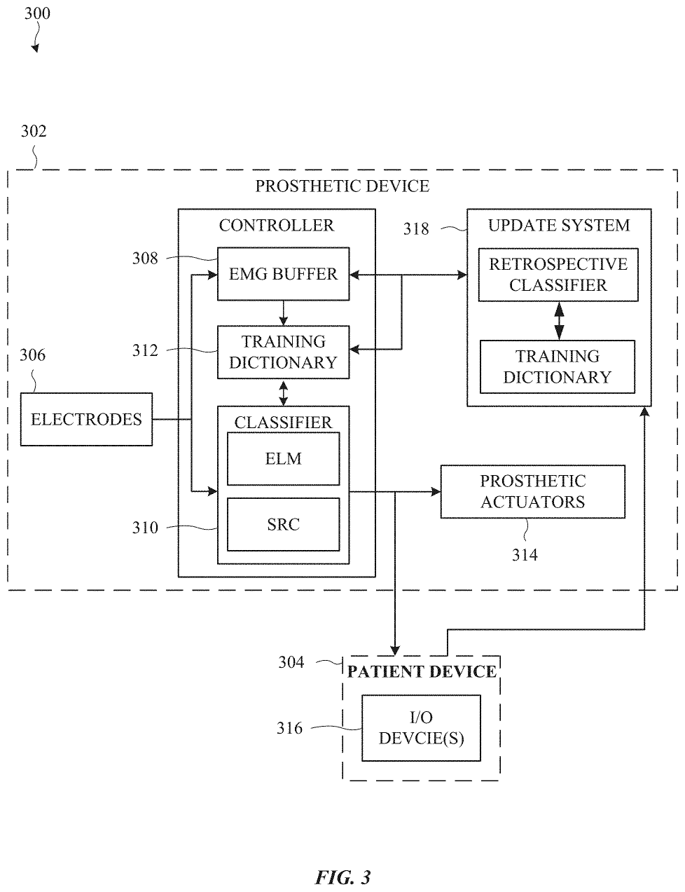

FIG. 3 shows an example process diagram 300 for adaptive control of a prosthetic device. The process flow 300 can include steps performed by a prosthetic device 302, a patient device 304 and communications between these devices. The prosthetic device 302 can be an example of the prosthetic devices described herein and the patient device 304 can be an example of the patient devices described herein.

The prosthetic device 302 can include one or more electrodes 306 which can be examples of the myoelectric sensors described herein. The electrodes 306 may sense myoelectric signals of the patient's residual limb and output myoelectric data indicative of the measured myoelectric activity to an electromyographic (EMG) buffer, which may store the myoelectric data for a period of time. In some cases, the myoelectric data may be stored until the classifier has processed the myoelectric data, the prosthetic actuators 314 have performed a movement and the patient has had an opportunity to provide feedback, for example, by indicating that an incorrect movement was performed. The electrodes 306 may also output the myoelectric data to the classifier 310.

The classifier 310 can include an ELM that takes detected myoelectric signal data as input and outputs one or more classifications and a confidence associated with each classification. The one or more classifications can correspond to a movement that the patient intends the prosthetic device to perform. Accordingly, in some cases, the control system can output control signals that cause a prosthetic device to perform a specific movement based on the classification of the ELM.

Additionally or alternatively, the classifier 310 can include an SRC. The SRC may be used to determine close calls between two or more movement classifications by the ELM. For example, the ELM may output two (or more) movement classifications that have similar confidence metrics. In these cases, the SRC can analyze the myoelectric data to determine which of the two movements was more likely intended by the patient.

The classifier 310 may interface with a training dictionary which can store training data for the ELM and/or the SRC. In some cases, the training data can include sub-libraries that correspond to different patient movements that have been trained by the classifier 310. The sub-library structure may allow training data for individual movement libraries to be updated independent of other movement data. Additionally or alternatively, the classifier (e.g., SRC) may access individual movement sub-libraries when classifying myoelectric data, which may increase the speed and efficiency of performing the classification functions as compared to accessing the entire movement library for all trained movements.

The output of the classifier can be used to send control commands to the prosthetic actuators 314. The prosthetic actuators 314 can include the components in a prothesis that generate motion such as hand motion(s) instructed by control commands from the controller. In some cases, the control commands can cause the prosthetic to perform a movement such as opening the hand, closing the hand, moving the hand to a specific position. In other cases, the control commands can include instructions for performing more complex operations such as a sequence of movements. For example, the control commands can instruct a sequence of movements to pick up an object and cause the prosthetic actuators 314 to perform a sequence of movements such as first opening the hand so that it can be positioned around and object and then closing the hand to grip the object.

In some cases, the prosthetic actuators 314 can include one or more sensors, which may be used to control the timing of the movements. In the sequence of opening a hand followed by closing of a hand to pick up an object, the sensors may be used to determine when the hand is ready to be closed. For example, the prosthetic actuators 314 may perform the first motion of opening the hand and then wait for feedback from one or more sensors indicating that the hand is positioned around an object. In response to the feedback, the prosthetic actuators 314 may perform the second movement of closing the hand.

The patient device 304 can be sent an indication of the movements performed by the prosthetic actuators 314. In some cases, the controller may send indications of the control commands to the patient device. Additionally or alternatively, the controller can send indications of alternative movements. For example, the classifier 310 may have identified two (or more) movements with high probabilities of being the intended movement of the patient. The classifier may select one of these movements as the intended movement. The controller may send an indication of the selected movement to the patient device and also send an indication of the next most probably movement that was not selected by the classifier 310. In some cases, the controller may send multiple alternative movements. Additionally or alternatively, the controller can send a determined probability that each movement was the intended movement of the patient.

In some cases, the prosthetic actuators 314 can communicate with the patient device 304. For example, the prosthetic actuators 314 may send an indication that a movement has performed to the patient device 304, which may trigger a feedback solicitation process for the performed movement at the patient device 304.

The patient device 304 can include an I/O device(s) as described herein, which can be used to output information to the patient and receive feedback from the patient. For example, the I/O devices may indicate a movement classification that was recently performed by the prosthetic actuators 314 (e.g., open hand) and provide an option for the patient to indicate whether that was the intended (correct) movement. In some cases, the I/O device may also output an indication of the next most probably movement (e.g., palm up) and provide an option for the patient to select this as an intend movement. In some cases, not receiving feedback from the patient device 304 may be interpreted by the prosthetic device 302 as the identified movement being the intended movement of a patient. In other cases, the patient selecting an alternative movement that is output by the I/O device 316 can be an implicit indication that the movement identified by the classifier 310 was not the intended movement and a update process can be initiated.

In response to the patient selecting an alternative movement, the patient device 304 can trigger an update process at the update system 318. In some cases, the patient device 304 sends an indication of the alternative movement selected by the patient to the update system 318. The updated system 318 can use this data to retrain the classifier so that myoelectric data that resulted in the misclassification (e.g., of open hand) will now result in the movement classification indicated by the patient (e.g., palm up) as described herein.

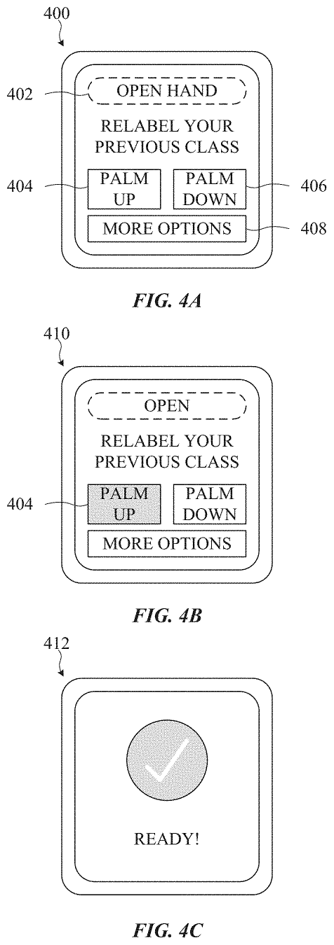

FIGS. 4A-4C show example user interfaces for providing patient input to a prosthetic system. The user interfaces may be displayed by a patient device such as a smartwatch, smartphone or other suitable electronic device as described herein.

FIG. 4A shows an example of a user interface instance 400 that may be presented to a patient after a movement is performed by the prosthetic device. The user interface instance 400 can include a first output 402 that displays an indication of the last command performed by the prosthetic device (e.g., “OPEN HAND”). The user interface instance 400 can also include a first option 404 and a second option 406 to relabel the previous movement. In some cases, the first and second options 404 and 406 may the next most probable movement classifications identified by the classification model.

FIG. 4B shows an example of a user interface instance 410 in response to a patient selecting the first option 404 to relabel the previous movement. In some cases, electronic device can include a touch-sensitive display as described herein and the patient may touch the area on the display corresponding to the first option 404. In other cases, the patient may select the first option 404 using other methods such as an input button, voice command, or using any other suitable input mechanism. In response, to the patient selection the user interface instance 410 may change a color, brightness, or other feature of the first option 404 to indicate that it was selected. The patient device may also send an indication of the selected option to the prosthetic device, which may initiate an update of the classification model as described herein.

FIG. 4C shows an example of a user interface instance 412 which may displayed after the classification model has been updated. This user interface instance 412 may provide confirmation to the patient, that their feedback was used to update the system and/or that the system is ready to receive additional myoelectric inputs.

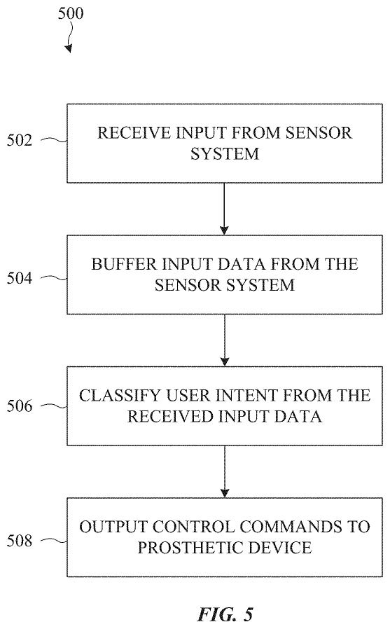

FIG. 5 shows an example process 500 for determining a control command for a prosthetic device from sensor input data. The process can be performed by the prosthetic devices described herein.

At 502, the process 500 can include receiving an input from a sensor system. In some cases, the input can be myoelectric input detected by one or more myoelectric sensors that are coupled to a patient. In other cases, the input can include other sensor data that is collected and used to control a downstream system or process. The received input can be processed data, which can include filtering, down sampling, digitizing, or otherwise modifying the raw sensor data to prepare it for analysis. The input data can correspond to signals measured over a time window, which could be a time window that captures changes myoelectric signals as a patient signals a movement that they would like to perform. Additionally or alternatively, the input can be a continuous or semi-continuous stream of data, which may be broken up into different time windows each of which may be analyzed.

At 504, the process 500 can include buffering input data from the sensor system. In some cases, the collected data may be stored while the data is classified and the movement is performed by the prosthetic device. Accordingly, if the patient indicates that a misclassification occurred, the buffered data can be retrieved and used to update the classification model. In some cases, the input data can be buffered until a patient is given an opportunity to correct a movement performed by the prosthetic device, which may be for a defined amount of time after the device has performed the movement and/or the patient provides a positive confirmation that the intended movement was performed.

In other cases, the input data may buffered for longer periods of time. For example, the classification model may misclassify the input data, but the patient may not immediately indicate that an unintended movement was performed. However, the patient may go back at a later time (e.g., end of the day) to indicate that specific movements were incorrect. Accordingly, the data may be buffered for longer periods of time to give the patient an opportunity to provide feedback at a later time. In some cases, the input data can be buffered for a period of time and then moved to longer term storage for later analysis. For example, if a patient does not provide any immediate feedback on a particular movement, the system may move the buffered data to a longer term storage and set a reminder to prompt the patient about the movement at a later time.

At 506, the process 500 can include classifying a patient intend from the receive input data. This can include providing the data as input to a classification model, as described herein. The classification model can include an ELM component that can be used to classify the input data and identify an intended movement of a patient. In some cases, the ELM can include a single layer feed forward artificial neural network that outputs one or more movement classifications for the input data along with a probability for each movement classification.

The classification model can be configured to compare the two (or more) movement classifications with the highest probabilities. If a difference between the probabilities of the two movement classification exceeds a threshold, the classification model can be configured to select the movement classification with the highest probability as the intended movement. If a different between the probabilities of the two movement classifications does not exceed the threshold, the classification model can be configured to perform additional analysis on the two (or more) most probably movement classifications.

In some cases, the classification model can perform a SRC on the data sets for the movement classes with the two highest probabilities. The SRC procedure can be performed using a reduced data library including data for the selected movements. The SRC procedure can determine the contributing vectors from each movement classification and their respective sparsity coefficients, which can be used to reconstruct the input data. The movement classification that minimizes the resulting residual can be chosen as the intended movement. Accordingly, the SRC procedure may be used in cases where the ELM identifies multiple movement classifications with similar probabilities.

At 508, the process 500 can include outputting control commands to the prosthetic device based on the intended movement that was identified by the classification model. In some cases, the control commands can cause an electromechanical actuated component, such as a prosthetic had to perform the intended movement.

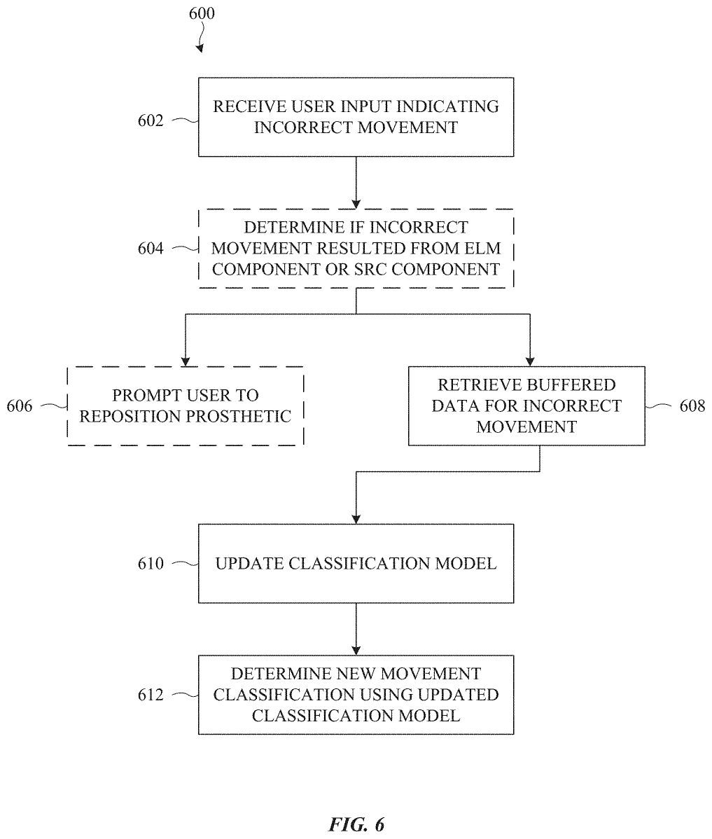

FIG. 6 shows an example process 600 for updating a control system based on a patient indicating an incorrect prosthetic movement. The process can be performed by the prosthetic system described herein.

At 602, the process 600 can include receiving a patient input indicating an incorrect movement classification. In some cases, the patient input can be in response to the prosthetic device performing a movement that was not intended by the patient as described herein. In other cases, the system may prompt the patient after the classification model is determined an intend movement, but before the movement has been performed by the prosthetic device. In these cases, the system may seek a movement confirmation prior to performing the movement.

The patient can provide their input to an electronic device such as a smartwatch, smartphone or other portable electronic device. In some cases, the electronic device for receiving patient input can be integrated with the prosthetic device. For example, the frame of the prosthetic device can include a touch screen interface, microphone, input button(s) or other input mechanism. The patient may provide input in a variety of ways. In some cases, the patient can interact with a touch-sensitive display as described herein. In other cases, the patient may provide voice input. In yet other examples, the patient can provide tactile feedback such as in the form of a tap, swipe and/or other gesture to a touch-sensitive input device such as a smartwatch or input device positioned on the prosthetic device.

In other cases, the patient may provide myoelectric feedback. For example, there may be specific types of and or intensity of myoelectric signals that a patient can generate and the classification model can identify with high accuracy. This could be a strong flexion of the residual limb or some other patient motion that the classification system can be distinguished with high accuracy and be used to provide feedback to the system. In this regard, a patient may be able to provide feedback using the myoelectric sensing system without needing to interact with another electronic device. In some cases, this type of detection can use additional sensors such as strain based sensing, force based sensing and so on which may help differentiate a patient's movement that is intended as feedback from myoelectric signaling of an intended movement for the prosthetic device.

At 604, the process 600 can include determining whether the incorrect movement resulted from the ELM component or the SRC component. In some cases, the movement classification may be made by the ELM, for example, when the a probability of one movement class is at least some threshold greater than a probability of any other movement class. In these cases, the system may be more confident in the movement classification, which may indicate that the classification error is due to a factor may be addressed without recalibration. For example, ELM classification errors may be due to improper positioning of the prosthetic device on the patient's arm, which may be more easily addressed by adjusting the prosthetic device as opposed to trying to update the classification model.

Classification errors that arise due to the SRC component may result from normal/unavoidable environmental variations that occur over time. Accordingly, in cases, where classification errors arise from the SRC component, it may be desirable to address these types of changes by updating the classification system. That is, these types of environmental changes may be longer term and updating the classification system may reduce the amount of errors that occur over time.

At 606, the process 600 can include prompting the patient to reposition the prosthetic device. Step 606 may be performed in response to determining that the error resulted from the ELM component. Once repositioned, the system can continue normal operation or perform a test to determine if the repositioning solved the issue. For example, after repositioning the prosthetic device, the patient may be instructed to perform the misclassified movement and the system can attempt to classify the intend movement a second time. The system can then determine whether the repositioning helped the issue based on the new movement classification.

At 608, the process 600 can include retrieving buffered data for the incorrect movement. As described herein myoelectric data that was generated from detected myoelectric signals can be buffered while the classification process is being performed. In response to the patient indicating an incorrect movement, the system can retrieve the myoelectric data, which may be used to update and/or retrain the classification model.

At 610, the process 600 can include updating the classification model using the retrieved myoelectric data and the patient feedback. An indication of the intended (correct) movement can be used to update the machine learning component. In some cases, this can include updating the data libraries associated with the SRC. For example, if the ELM identified two potential movements as the intended movement and the SRC incorrectly output one of those movements as the intended movement, then the SRC data libraries associated with the two movements can be updated to achieve the correct movement output.

The SRC data libraries can be updated so that the same myoelectric data that originally lead to the incorrect movement classification now leads to the correct movement classification. In some cases, this can include removing a portion of the data stored in the SRC libraries for the two movements and updating this portion based on the detected myoelectric signals and the correct movement as indicated by the patient. In other cases, the data in the SRC libraries can be replaced, for example, by using the detected myoelectric data to regenerate library data for the correct movement.

Additionally or alternatively, the ELM can be updated based on the detected myoelectric data and the patient feedback. For example, the myoelectric data can be used to retrain the ELM classification model so that the detected myoelectric data is associated with the correct prosthetic movement that was indicated by the patient. Using an ELM can have the advantage of quick retraining times allowing the ELM classification model to updated in real-time (e.g., in response to the patient feedback), while minimizing any downtown for the prosthetic system. Additionally, the use of the ELM to reduce the data set to a limited number of movement classifications allows the SRC to operate more quickly, to support real-time movement classification.

At 612, the process can include determining a new movement classification using the updated classification model. In some cases, the update can occur in real time and be implemented into the system as soon as the SRC library has been updated and/or the ELM model has been retrained. The discrete movement data in the SRC library may only require a portion of the library to be updated, which may facilitate quick updates with minimal downtime. Additionally, the structure of the ELM component also allows fast updating of the ELM as compared to other types of neural networks. Accordingly, the prosthetic device may update the classification model without the patient noticing or needing to perform any additional actions other than the initial feedback that an incorrect movement was performed.

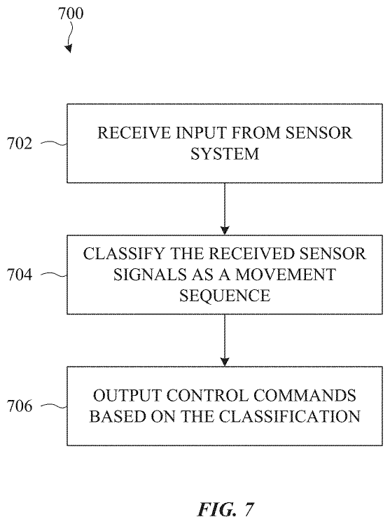

FIG. 7 shows an example process 700 for determining a movement sequence from myoelectric signals. The process can be performed by the prosthetic system described herein.

At 702, the process 700 can include receiving input from the sensor system. In some cases, the input can be myoelectric input detected by one or more myoelectric sensors and corresponding to a movement sequence. The received input can be processed, which can include filtering, down sampling, digitizing, or otherwise modifying the raw sensor data to prepare it for analysis. The input signals can correspond to signals measured over a time window, which could be a time window that captures changes myoelectric signals as a user signals a movement sequence that he or she intends to perform. Additionally or alternatively, the input can be a continuous or semi-continuous stream of data, which may be broken up into different time windows each of which may be analyzed.

At 704, the process 700 can include classifying the received sensor signals as a movement sequence. The classification model can be trained to include one or more movement sequences for a user. For example, a user may routinely perform specific sequences of movements such as a handshake, grabbing an object or other movement sequences in which multiple joints are moving simultaneously or near simultaneously. In some cases, myoelectric signals for movement sequences may occur as a distinct pattern that may not correlate well with discrete movements such as a palm open or palm up procedure. For example, in individuals with a full arm and hand, myoelectric signals may terminate in different regions or muscle groups along the limb which effectively spreads out a signal density. However, in an individual with a partial limb, the myoelectrical signals may be more highly concentrated in a localized region of the arm. For example, nerves that would have extended into the hand my now terminate at an upper portion of the user's limb. As a user attempts to perform a complex movement sequence, myoelectric signals that would have terminated in different regions, such as a hand, wrist, elbow, all terminate a portion of the residual limb. This may create a unique myoelectric pattern that does not correspond closely to any individual movement.

The myoelectric data corresponding to a movement sequence can be input into the classification model, as described herein. The classification model may output a movement sequence class. At 706, the process 700 can include outputting control commands to an electromechanical device based on the movement sequence classification. The control commands can include multiple movements and/or a sequence and timing information associated with the movements.

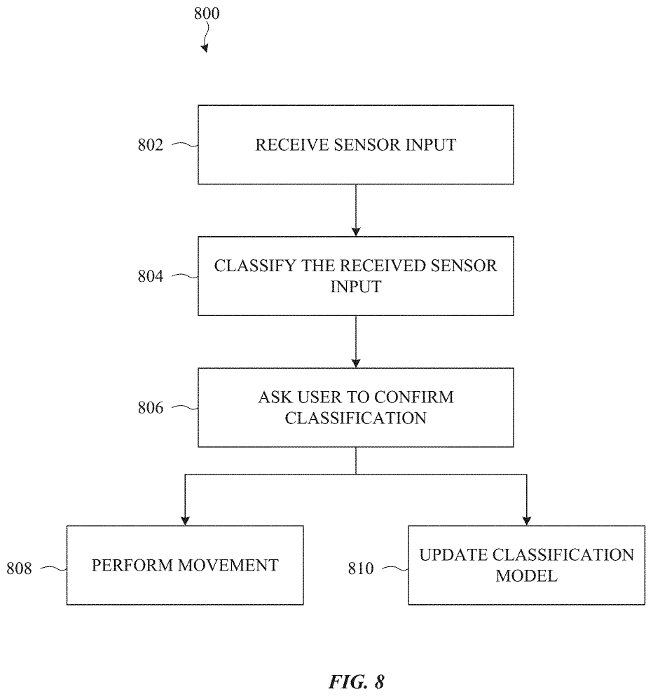

FIG. 8 shows an example process 800 for a user confirmation process prior to performing an identified movement. The process can be performed by the prosthetic system described herein.

At 802, the process 800 can include receiving sensor input, which can be an example of the receiving sensor input operations described herein (e.g., operations 502 and 702). At 804, the process 800 can include classifying the received sensor input, which can be an example of the classification procedures described herein.

At 806, the process 800 can include asking a user to confirm the classification. In some cases, the system can implement a proactive confirmation to have the user confirm whether their intended movement has been identified correctly. This proactive classification can be performed for specific types of movements. For example, a prosthetic hand may be configured to perform a strong grip operation. However, if performed at the wrong time the strong grip movement may break an object or cause other damage. Accordingly, the system may be configured to confirm that the user intends to perform the strong grip function prior to sending any commands to the electromechanical actuators.

In other cases, confirmation step can be threshold based. For example, the classification model may determine two different movements that have similar probabilities. If a difference between the probabilities is below a threshold, instead of performing one of the movements and waiting for feedback, the system may prompt the user to confirm or select one of the movements prior to performing the movement. At 808, the process 800 can include performing the movement that was selected by the user.

At 810, the process 800 can include updating the classification model base on proactive confirmation received from the user. As a user confirms or selects one of the movements, this data can be used to modify the training library for the SRC component and/or retrain the ELM component. This updating can result in an increase in the probability of the movement from the classification model based on the same user input. In some cases, if the user confirms an appropriately classified movement a defined number of times, then the system may start automatically performing that movement.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art that many modifications and variations are possible in view of the above teachings.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art that many modifications and variations are possible in view of the above teachings.

What is claimed is:

1. A prosthetic system comprising:
a prosthetic device comprising:
one or more sensors configured to detect myoelectrical signals of a patient; and
a controller configured to:
provide the array of movement data as input to a classification model, the array of movement data based on the detected myoelectric signals;
receive, from the classification model:
a first classification corresponding to a first movement to be performed by the electromechanical hand;
a first confidence metric associated with the first classification; and
a second classification corresponding to a second movement to be performed by the electromechanical hand; and
a second confidence metric associated with the second classification, the second confidence metric lower than the first confidence metric; and
transmit, to the electromechanical hand, a control command to perform the first movement; and
an electronic device configured to:
receive, from the prosthetic device, an indication of the first and second movements;
output, a selectable interface element for indicating the second movement as the intended movement of the patient; and
in response to the patient selecting the user interface element, causing the classification model to be updated.

2. The prosthetic system of claim 1, wherein the update to the classification model causes the classification model, in response to receiving the array of movement data, to output the second label with a higher confidence than the first label.

3. The prosthetic system of claim 1, wherein the prosthetic device further comprises:
a frame configured to couple to a limb of a patient; and
the one or more sensors are coupled to the frame.

4. The prosthetic system of claim 1, wherein the prosthetic device further comprises an interface for coupling to an electromechanical hand.

5. The prosthetic system of claim 1, wherein the electronic device comprises:
a touch-sensitive display; and
the touch-sensitive display is configured to display the selectable interface element.

6. The prosthetic system of claim 5, wherein the selectable interface element is selected via a patient interaction with the touch-sensitive display.

7. A prosthetic system as shown and described.

8. A method of operating a prosthetic system as shown and described.
